(19) United States
(12) Patent Application Publication
(10) Pub. No.: US 2005/0063932 A1
(43) Pub. Date: Mar. 24, 2005

(54) Skin care compositions including hexapeptide complexes and methods of their manufacture

(76) Inventors: Natalie Dilallo, Los Angeles, CA (US); Alyson Galderisi, Wayne, NJ (US)

Correspondence Address: CISLO & THOMAS, LLP, 233 WILSHIRE BLVD, SUITE 900, SANTA MONICA, CA, 90401-1211, US

(21) Appl. No.: 10/918,135

(22) Filed: Aug. 13, 2004

Related U.S. Application Data
(60) Provisional application No. 60/495,574, filed on Aug. 14, 2003.

(51) Int. Cl. A61K 7/06; A61K 7/11

(52) U.S. Cl. 424/70.14; 424/74

(57) ABSTRACT

Skin care compositions disclosed herein include, at a minimum, safe and effective amounts of at least one wrinkle reduction agent and a natural exfoliating complex. The skin care compositions provide natural skin exfoliation, reduce fine lines and wrinkles, and improve skin elasticity and firmness.

### CROSS REFERENCE TO RELATED APPLICATIONS

This application claims the benefit of U.S. Provisional Application No. 60/495,574, filed on Aug. 14, 2003, and whose entire contents are hereby incorporated by reference.

### BACKGROUND

1. Field of the Invention

The present invention relates to cosmetic compositions and, in particular, to multi-purpose skin care compositions.

2. Description of the Related Art

Various skin care products such as creams, lotions, bar soaps and gels are commercially available to cleanse, moisturize, exfoliate and minimize wrinkling of the skin. Typically, these skin care products are designed for a singular purpose. In order to achieve multiple benefits, an individual may have to use more than one skin care product. Recently, skin care products have been developed that may have more than one use such as a combination cleanser and moisturizer. However, there can some difficulty in producing multi-use products as the physical properties of each individual component may not be compatible with one another. Also, these multi-purpose products may not be as effective when used alone. Accordingly, there remains a need for multi-purpose skin care products that can provide multiple skin care benefits.

### SUMMARY

Exemplary embodiments disclosed herein are directed to skin care compositions that include one or more ingredients that provide for natural skin exfoliation, reduce fine lines and wrinkles and improve skin elasticity and firmness. According to a first embodiment, the skin care composition is composed of a safe and effective amount of at least one anti-wrinkling agent and a safe and effective amount of a natural exfoliating complex.

In another embodiment, the anti-wrinkling agent is a hexapeptide. In another embodiment, the hexapeptide is an acetyl hexapeptide-3.

In yet another embodiment, the natural exfoliating complex is composed of ahnfeltia cocinna. In another exemplary embodiment the natural exfoliating complex also includes butylene glycol and glycosaminoglycans.

In yet another embodiment, the skin care compositions include a safe and effective amount of a hexapeptide or a hexapeptide complex, preferably in combination with one or more of the following ingredients: Willow Bark Extract, APT (glycosaminoglycans, water, butylenes glycol and Ahnfeltia extract), Moist 24 (Imperate Cylindrica (Root) Extract, Water, Glycerin, PEG-8, and Carbomer), Skin Flux, Polyolprepolymer-15, Dipalmitoyl Hydroxyproline (DPHP), Deliner (zea mays extract), and Oat Beta Glucan.

In yet another embodiment, the compositions contain at least three ingredients selected from the following group: a hexapeptide complex, Willow Bark Extract, APT (glycosaminoglycans, water, butylenes glycol and Ahnfeltia extract), Moist 24, Skin Flux, Polyolprepolymer-15, Dipalmitoyl Hydroxyproline (DPHP), Deliner (zea mays extract), and Oat Beta Glucan.

In other embodiments, the skin care composition may include one or more additional components such as, but not limited to, conditioning agents, skin protectants, antioxidants, sunscreen actives, cleansing agents, cosmetic soothing aids, or the like.

### DETAILED DESCRIPTION

The detailed description set forth below is intended as a description of exemplary embodiments and is not intended to represent the only forms in which the exemplary embodiments may be constructed and/or utilized. The description sets forth the functions and the sequence of steps for constructing and/or operating the exemplary embodiments. However, it is to be understood that the same or equivalent functions and sequences may be accomplished by different exemplary methods are also intended to be encompassed within the spirit and scope of the invention.

As used herein, “safe and effective amount” means a sufficient amount of a compound, composition or other material described by this phrase to significantly induce a positive modification in the condition being treated, but low enough to avoid undue side effects (e.g., significant skin irritation or sensitization), within the scope of sound judgment of the skilled person. The safe and effective amount of the compound, composition or other material may vary with the particular skin being treated, the age and physical condition of the biological subject being treated, the severity of the condition, the duration of treatment, the nature of concurrent therapy, the specific compound, composition, or other material employed, the particular cosmetically acceptable topical carrier utilized, and the factors within the knowledge and expertise of the skilled person.

Exemplary embodiments disclosed herein are directed to skin care compositions that include one or more ingredients that provide a plurality of skin care benefits such as, but not limited to, exfoliating the skin naturally, reducing fine lines and wrinkles and improving skin elasticity and firmness.

According to a first embodiment, the skin care composition is composed of a safe and effective amount of at least one anti-wrinkling agent and a safe and effective amount of a natural exfoliating complex.

In another embodiment, the anti-wrinkling agent is a hexapeptide. In another embodiment, the hexapeptide is an acetyl hexapeptide-3.

In yet another embodiment, the natural exfoliating complex is composed of ahnfeltia cocinna. In another exemplary embodiment the natural exfoliating complex also includes butylene glycol and glycosaminoglycans.

In yet another embodiment, the skin care compositions include a safe and effective amount of a hexapeptide or a hexapeptide complex, preferably in combination with one or more of the following ingredients: Willow Bark Extract, APT (glycosaminoglycans, water, butylenes glycol and Ahnfeltia extract), Moist 24 (Imperate Cylindrica (Root) Extract, Water, Glycerin, PEG-8, and Carbomer), Skin Flux, Polyolprepolymer-15, Dipalmitoyl Hydroxyproline (DPHP), Deliner (zea mays extract), and Oat Beta Glucan.

In yet another embodiment, the compositions contain at least three ingredients selected from the following group: a hexapeptide complex, Willow Bark Extract, APT (glycosaminoglycans, water, butylenes glycol and Ahnfeltia extract), Moist 24, Skin Flux, Polyolprepolymer-15, Dipalmitoyl Hydroxyproline (DPHP), Deliner (zea mays extract), and Oat Beta Glucan.

In other embodiments, the skin care composition may include one or more additional components such as, but not limited to, conditioning agents, skin protectants, antioxidants, sunscreen actives, cleansing agents, cosmetic soothing aids, or the like.

The hexapeptide complex is an anti aging ingredient that is chemically combined from naturally derived amino acids that minimizes and softens fine lines. It is an excellent wrinkle reducer of the fine to medium depth “expression” lines, and specifically targets the repeated, biomechanical, muscular contractions of facial expressions such as laughing, squinting, frowning, etc. by functioning to reduce the intensity of these muscle contractions that overtime engrave wrinkles in the skin. It also helps to relax muscles, as opposed to paralyzing them, like botox, thereby reducing the appearance of wrinkles without toxicity and without the loss of natural expression. Thus, it offers an injection free, non-toxic alternative to botox. The hexapeptide complex is preferably an acetyl hexapeptide-3. Depending on the application of the skin care composition, the amount of hexapeptide and other ingredients used may be varied. The skin care composition may include hexapeptide complexes having hexapeptide concentrations ranging from approximately 0.00001 to approximately 15% w/w, preferably from 0.01% w/w to 10% w/w, and more preferably from 0.1 to 5%.

The hexapeptide component of the hexapeptide complex has been clinically shown to minimize wrinkling of the skin. As those skilled in the art will appreciate, wrinkling of the skin can be attributed to natural aging and overexposure to the sun. Although the mechanism of wrinkle formation is not completely known, studies have shown that visible fine wrinkles may be attributable to a reduction in the number and size of elastic fibers in the papillary dermis, an atrophy of the dermis and a reduction in subcutaneous adipose tissue. With respect to coarse wrinkles, studies have shown that these wrinkles may be attributed to excessive deposition of abnormal elastic elements in the upper dermis and thickening of the skin.

The hexapeptide component has been shown to reduce wrinkle formation by reducing the release of catecholamines and limiting the production of SNARE complexes, which are membrane proteins that regulate neurotransmitter release. Studies have shown that minimizing the production of the SNARE complexes decreases the formation of coarse wrinkles. Additionally, the hexapeptide compound has been shown to minimize the overproduction and release of catecholamines, which are attributable to wrinkle formation and fine wrinkles.

APT or APT GC is a natural, mild, exfoliation medium that increases cellular turnover (fibroblast proliferation) by approximately 18% within about 4 weeks, without the over stimulation commonly caused by the use of alpha hydroxyacids or retinal that commonly irritated and cause hyperplasia (an uneven thickening of the skin in an erratic formation). Thus, cells rebuild more slowly and in a more uniform and organized manner like a brick and mortar fashion. This slow rebuilding process ensures a more perfected, symmetrical, cell alignment, and therefore, a more uniform skin health, firmness, elasticity, and tonality or skin opacity. Raw material cell culture studies show that the newly rebuilt cells possess a high water binding capacity that provide intense, immediate, super hydration. Also, the APT can increase the density of the collagen bundles of the upper dermis, which can lead to greater skin elasticity. The Ahnfeltia Concinna component of APT provides for the natural exfoliation of skin. In contrast to prior art exfoliants such as AHA, APT GC can permit the uniform exfoliation of the skin. That is, while AHA can increase skin cell turnover (i.e. exfoliate the top layer(s) of skin), it is particularly harsh and may cause “skin shock” that may cause redness and irritation of the skin. Accordingly, the underlying skin layers may regenerate at different rates leading to uneven skin appearance. Furthermore, AHA can have different rates of penetrating the skin that may also contribute to blotchiness that can be associated with exfoliation procedures.

In contrast to prior art exfoliants, APT can improve skin tone by approximately 15% after 4 weeks of use. The APT component can be more uniformly absorbed by the skin to reduce blotchiness. Also, pore size can be reduced by approximately 35% and skin texture can be improved by approximately 42% after 4 weeks of use. Furthermore, because the APT component does not utilize harsh chemicals, redness and irritation can be reduced. With respect to skin redness, the skin care composition can improve skin redness (i.e., lessen skin redness) by approximately 25% after 2 weeks and by approximately 33% after 4 weeks of use. With respect to skin irritation, the skin care composition can decrease skin irritation by approximately 9% after 2 weeks and approximately 23% after 4 weeks of use. The concentration of APT contained in the formulations preferably ranges from 0 to approximately 5% w/w and more preferably from approximately 0.01% w/w to approximately 2% w/w.

The willow bark extract is a natural exfoliating extract that can increase the cellular renewal capability of the formulations better than salicylic acid and with less irritation. The willow bark extract also creates a general improvement in the skin's appearance that results in smother skin and a reduction in fine lines and wrinkles. The willow bark extract that is used in the compositions of the present invention is preferably white willow bark which has an irritation factor comparable to that of glycerin tat registers at an almost non-existent level. It is therefore a well suited exfoliation vehicle for aging, dry, dehydrated, and sensitive skin. The amount of willow bark extract used in the formulations of the present invention ranges from approximately 0 to 5% w/w, and more preferably from between 0.2 and 2.5%.

The Moist 24 component is a plant based, twenty four hour, long lasting moisturizer that is beneficial for the continuous hydration of aging skin. It is also excellent for dry, dehydrated skin that is deficient in potassium, since potassium balances the acid/alkaline system in the body and works with sodium to regulate the skin's water balance among many other functions. The Moist 24 used in the formulations of the present invention preferably ranges from between approximately 0 and 3% w/w, and more preferably between approximately 1 and 0.25%.

DPHP reduces wrinkles caused by physiological aging. It defends against both natural and photo-induced skin aging factors, including weakening of the collagen fiber network, degrading of the dermal matrix and the dermal/epidermal junction, and free radical damage that has mediated to skin cells within the fibroblasts. It further helps to prevent resulting skin aging effects including flaccid, lose skin, loss of tone and firmness, development of wrinkles and crow's feet, skin structural loss and moisture content. Additionally, it delivers a complex, triple action effect upon aging including anti-wrinkle, effects, long lasting lipophilic moisturizing activity, and skin firming by stimulating pro-collagen production to protect the extra cellular dermal matrix, protecting the elastin in the skin, and protecting fibroblasts against free radicals. The preferred amount of DPHP used in the formulations of the present invention ranges between 0 and approximately 1% w/w.

The Skin Flux component effectively enhances skin moisture because of its unique skin identical lipid concentration. It improves skin barrier function and therefore provides exceptional protection for the skin. It further enhances the delivery and exchange of skin lipids to facilitated “lipid dry” skin conditions as opposed to “water dry” conditions, and is ideal for aging, dry, dehydrated and sensitive skin that in most cases, commonly experiences depleted lipid content. The preferred amount of Skin Flux used in the compositions of the present invention ranges from 0 to approximately 5%, and more preferably from between 1 and 2% w/w.

Polyolprepolymer-15 is a component which improves the deposition of actives in the upper layers of the skin and creates a well suited delivery systems for the active ingredients in the compositions of the present invention. It ensures a slow, but optimum timing of the deposition of the active components due to its chemical structure and molecular weight, creating within the stratum cornium a long lasting liquid reservoir for release of various materials in the skin. The prepolyolprepolymer-15 component is particularly ideal for compositions which have a high amount of active ingredients, since it slows their penetration in a timed release manner that does not shock the skin.

As the skin ages, the prefect integrity of the extra-cellular matrix, which is a combination of collagen, elastin, and glycosaminoglycans, undergoes alterations causing wrinkles to appear. The Deliner component stimulates fibronectin synthesis, which is the webbing of the extra-cellular matrix, which in turn, enables fibroblast to migrate to those areas of the matrix that have been altered or badly damaged. As a result, fibroblastic synthesis is reactivated and reforms the extra-cellular matrix. Ultimately the repaired matrix provides support and firmness to the skin tissue causing wrinkles to fade. The preferred amount of Deliner used in the compositions of the present invention may range from 0 to approximately 2% w/w, and is more preferably around 1%.

Oat Beta Glucan is a natural, soothing plant derivative which has anti-inflammatory properties. This component increases collagen synthesis and cell renewal, reduces fine lines and wrinkles, protects the skin from UV damage, and serves as a protective film-forming agent. The preferred amount of Oat Beta Glucan used in the compositions of the present invention may range from approximately 0 to 5% w/w, and more preferably between 1 and 2%.

The combination of ingredients of the skin care compositions of the present invention are believed to provide synergistic benefits which are otherwise not available with any one of the components used by itself. Clinical evaluations based on the use of the hexapeptide complex itself evidenced a reduction in fine lines and wrinkles, including laugh lines, frown lines, and smile lines, by approximately 13% after 2 weeks and approximately 33% in 4 weeks of use. It was further observed that 100% of participants experienced a reduction in under-eye fine lines and wrinkles on the average of approximately 16% after 4 weeks and approximately 52% after 8 weeks of use. Additionally, an increase in moisture content by 37% after 15 minutes of use was observed.

It is further believed that the compositions of the present invention may improve skin complexion causing the skin to be more flawless and even-toned. Additionally, it is believed that the use of the compositions may increase cellular turnover by 100% with minimal irritation after approximately 2 to 4 weeks of use.

Furthermore, in other exemplary embodiments, the skin care composition may include other components including, but not limited to, conditioning agents, skin protectants, antioxidants, UV absorbing agents, sunscreen actives, cleansing agents, viscosity modifying agents, film formers, emollients, surfactants, solubilizing agents, preservatives, fragrance, chelating agents, foaming or antifoaming agents, opacifying agents, stabilizing agents, pH adjustors, absorbents, anti-caking agents, slip modifiers, various solvents, solubilizing agents, denaturants, abrasives, bulking agents, emulsion stabilizing agents, suspending agents, colorants, binders, conditioning agent-emollients, surfactant emulsifying agents, biological products, anti-acne actives, anti-wrinkle and anti-skin atrophy actives, skin barrier repair aids, cosmetic soothing aids, topical anesthetics, artificial tanning agents and accelerators, skin lightening actives, antimicrobial and antifungal actives, sebum stimulators, sebum inhibitors, humectants, and/or combinations thereof.

Non-limiting examples of sunscreens which may be useful in the skin care compositions include 4-N,N-(2-ethylhexyl)methylaminobenzoic acid ester of 2,4-dihydroxybenzophenone, 4-N,N-(2-ethylhexyl)methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone, 4-N,N-(2-ethylhexyl)-methylaminobenzoic acid ester of 4-(2-hydroxyethoxy)dibenzoylmethane, 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, and mixtures thereof. Other useful sunscreens include aminobenzoic acid (PABA), benzylidene camphor, butyl methoxy dibenzoyl methane, diethanolamine p-methoxycinnamate, dioxybenzone, ethyl dihydroxypropyl (PABA), glyceryl aminobenzoate, homomenthyl salicylate, isopropyl dibenzoyl methane, lawsone and dihydroxyacetone, menthyl anthranilate, methyl anthranilate, methyl benzylidene camphor, octocrylene, octyl dimethyl (PABA), octyl methoxycinnamate, oxybenzone, 2-phenylbenzimidazole-5-sulfonic acid, red petrolatum, sulisobenzone, titanium dioxide, triethanolamine salicylate, zinc oxide, and mixtures thereof. Exact amounts of sunscreens that can be employed will vary depending upon the sunscreen chosen and the desired Sun Protection Factor (SPF) to be achieved.

The exemplary embodiments of the skin care compositions disclosed herein may also be used alone or in combination with one or more products. For instance, a skin care system may include, but is not limited to, a day cream, night cream, eye serum, mask, cleanser, and an exfoliant. The following examples are directed to various exemplary embodiments of the skin care composition and methods of manufacturing those compositions.

### EXAMPLE 1

The composition of this example includes sun-screening agents and is preferably used as a daytime facial cream. The ingredients included in the composition are listed in Table 1:

TABLE 1

| Seq. | INCI Name | Trade Name | % w/w |
|---|---|---|---|
| 1 | Water | Water | 55.49 |
| 2 | Carbomer | Carbopol 940 | 0.40 |
| 3 | Glycerin | Glycerin 96% | 1.00 |
| 3 | Trisodium EDTA | Sequestrene NA3T | 0.10 |
| 3 | Methylparaben | Methylparaben | 0.25 |
| 3 | Propylparaben | Propylparaben | 0.10 |
| 4 | PEG-8/SMDI Copolymer | Polyoprepolymer-15 | 1.00 |
| 5 | Octyldodecyl Neopentanoate | Elefac I 205 | 5.00 |
| 5 | Octinoxate | Parsol MCX | 7.50 |
| 5 | Oxybenzone | Benzophenone-3 | 3.00 |
| 5 | Glyceryl Stearate (and) PEG-100 Stearate | Lipomulse 165 | 2.50 |
| 5 | Cetyl Alcohol | Lipocol C | 1.50 |
| 5 | Caprylic/Capric Triglyceride | Captex 300 | 2.00 |
| 5 | Octisalate | Escalol 587 | 5.00 |
| 5 | Cetearyl Alcohol (and) Ceteareth-20 | Lipowax D | 1.50 |
| 5 | Tricaprin | Captex 1000 | 0.50 |
| 5 | Dimethicone | Dow Corning | 0.25 |
| | | 200/100 cts Fluid | |
| 5 | Dipalmitoyl Hydroxyproline | Sepilift DPHP (Seppic) | 1.00 |
| 6 | Water | Water | 1.00 |
| 6 | Triethanolamine | TEA 99% | 0.40 |
| 7 | Phenoxyethanol | Phenoxyethanol | 1.00 |
| 8 | Caprylic/Capric Triglyceride (and) Cucumis | Natural Melon Blend Aroma | 1.50 |
| | Melo (Melon) Extract (and) Musa Sapientum | XX-000 981 (Ungerer) | |
| | (Banana) Fruit Extract (and) Vitis Vinifera | | |
| | (Grape) Fruit Extract (and) Iris Florentina Root | | |
| | Extract (and) Hibiscus Abelmoschuus | | |
| 9 | Water (and) Hyaluronic Acid | Hyasol 1% (Centerchem) | 1.00 |
| 9 | Acetyl Hexapeptide 3 | Argireline (Centerchem) | 5.00 |
| 9 | Water (and) Butylene Glycol (and) Ahnfeltia | APT (Centerchem) | 2.00 |
| | Concinna Extract (and) Glycosaminoglycans | | |
| 9 | Lecithin (and) Magnesium Ascorbyl Phosphate | Oxysomes (Barnet) | 0.01 |
| | (and) Tocopherol | | |

With reference to Table 1, the composition of Example 1 is manufactured using a stainless steel, jacketed main kettle, equipped with sweep agitation and a Lightning® mixer. The Seq. #1 water is heated to approximately 78-80° C. under mixing or agitation. Under good agitation, the Seq. #2 Carbomer powder is slowly added by sprinkling slowly and evenly across the agitating water. Care should be taken not to aerate the batch. The batch is then checked to assure that it is free from any lumps, then cooled to approximately 76-78° C. The remaining Seq. #3 materials are added to the batch under adequate agitation. Just prior to the addition of the Seq. #5 materials, the Seq. #4 Copolymer is added to the water phase batch in the main tank under good agitation. In a stainless steel side kettle, the Seq. #5 materials are combined and heated to approximately 80-82° C., under good agitation. At proper temperatures, the Seq. #5 combination is added to the Seqs. 1, 2, 3, and 4 materials in main tank with good agitation. The temperature and agitation is maintained until the emulsion is stabilized. Thereafter, the batch is cooled to approximately 70° C. and the agitation setting is switched to sweep as the batch thickens to avoid air entrapment. At approximately 70° C., the premixed Seq. #6 materials are added, and the batch is cooled. The Seq. 7 material may then be added. At approximately 42° C., the Seq. #8 material is added. The Seq. #9 materials are then added, one at a time, being sure batch is uniform before proceeding. Thereafter, the batch is cooled to approximately 25° C. A standard may be established by producing several batches which are similar (e.g. three batches). Once a standard is made, top and bottom samples may be taken from each subsequent batch for comparison to the standard.

According to an alternate procedure for preparing the composition of Example 1, the Seq. #1 water is heated in the main kettle to approximately 80-82° C. while mixing. Under good agitation, the Seq. #2 carbomer powder is then sprinkled slowly and evenly across the water. Care should be taken not to aerate the batch. Alternatively, the carbomer powder may be added prior to heating the water. The Seq. #3 materials are then added to the main kettle, and the batch is mixed well. Once the batch is free from lumps, it is cooled to 76-78° C. The Seq. #4 material, which may be heated to approximately 80-82° C., is then added to the batch in the main kettle under adequate agitation. Once the emulsion is well formed and stabilized, the batch is allowed to cool to approximately 70° C. The premixed Seq. #5 materials are then added to the batch. The mixing type and speed are adjusted as necessary as the batch thickens. The Seq. #6 materials are then added. The batch is then cooled to 50° C. and the Seq. #7 material is added to the batch. The batch is cooled to 42° C. The Seq. #8 materials are then added, one at a time, mixing well between additions, and reaching uniformity before proceeding. The batch is then cooled to approximately 35° C. The Seq. #9 materials are then added one at a time, mixing well between additions, and reaching uniformity. The batch is then cooled to room temperature, and compared to the standard.

### EXAMPLE 2

The composition of this example is preferably used as a nighttime facial cream and includes the following ingredients as listed in Table 2:

TABLE 2

| Seq. | INCI Name | Trade Name | % w/w |
|---|---|---|---|
| 1 | Water (Aqua) | Water | 53.84 |
| 2 | Carbomer | Carbopol 940 | 0.35 |
| 3 | Glycerin | Glycerin 96% | 3.00 |
| 3 | Trisodium EDTA | Sequestrene NA3T | 0.10 |
| 3 | PEG-8/SMDI Copolymer | Polyoprepolymer-15 | 1.00 |
| 3 | Methylparaben | Methylparaben | 0.25 |
| 3 | Propylparaben | Propylparaben | 0.10 |
| 4 | Isostearyl Palmitate | Protachem ISP | 3.00 |
| 4 | Glyceryl Stearate (and) PEG-100 Stearate | Lipomulse 165 | 2.50 |
| 4 | Squalane | Squalane | 2.00 |
| 4 | Cetyl Alcohol | Lipocol C | 1.00 |
| 4 | Caprylic/Capric Triglyceride | Captex 300 | 10.00 |
| 4 | Cetearyl Alcohol (and) Ceteareth-20 | Lipowax D | 2.00 |
| 4 | Cetyl Lactate | Ceraphyl 28 | 0.50 |
| 4 | Dimethicone | Dow Corning 200/100 cts | 0.50 |
| | | Fluid | |
| 4 | Dipalmitoyl Hydroxyproline | Sepilift DPHP (Seppic) | 1.00 |
| 5 | Water | Water | 1.00 |
| 5 | Triethanolamine | TEA 99% | 0.35 |
| 6 | Polyacrylamide (and) C13-14 Isoparaffin | Sepigel 305 | 0.25 |
| | (and) Laureth-7 | | |
| 7 | Cyclomethicone | Dow Corning 345 Fluid | 1.00 |
| 8 | Caprylic/Capric Triglyceride (and) Cucumis | Natural Melon Blend Aroma | 1.50 |
| | Melo (Melon) Extract (and) Musa Sapientum | xx-000981 | |
| | (Banana) Fruit Extract (and) Vitis Vinifera | | |
| | (Grape) Fruit Extract (and) Iris Florentina | | |
| | Root Extract (and) Hibiscus Abelmoschuus | | |
| 8 | Cucumis Melo (Melon) Extract | Crodarome Liquid Melon | 0.50 |
| | | Extract (Croda) | |
| 9 | Phenoxyethanol | Phenoxyethanol | 1.00 |
| 9 | Water (and) Hyaluronic Acid | Hyasol 1% (Centerchem) | 1.00 |
| 9 | Acetyl Hexapeptide 3 | Argireline (Centerchem) | 10.00 |
| 9 | Imperate Cylindrica (Root) Extract (and) | Moist 24 (Croda) | 0.25 |
| | Water (and) Glycerin (and) PEG-8 (and) | | |
| | Carbomer | | |
| 9 | Glycosaminoglycans (and) Water (and) | APT (Centerchem) | 2.00 |
| | Butylene Glycol (and) Ahnfeltia Concinna | | |
| | Extract | | |
| 9 | Lecithin (and) Magnesium Ascorbyl | Oxysomes (Barnet) | 0.01 |
| | Phosphate (and) Tocopherol | | |

With reference to Table 2, the composition of Example 2 is prepared by heating the Seq. #1 water in a stainless steel, jacketed main kettle, equipped with sweep agitation and a Lightnin® mixer, to approximately 80-82° C. while mixing. Under good agitation, the Seq. #2 carbomer powder is sprinkled slowly and evenly across the water. Care should be taken not to aerate the batch. Alternatively, the carbomer powder may be added prior to heating the water. The Seq. #3 materials are then added to the main kettle, and the batch is mixed well. Once the batch is free from lumps, it is cooled to 76-78° C. The Seq. #4 materials are combined in a stainless steel side kettle, under agitation, and heated to approximately 80-82° C. The Seq. #4 mixture is then slowly added to the batch in the main kettle under adequate agitation. Once the emulsion is well formed and stabilized, the batch is allowed to cool to approximately 70° C. The premixed Seq. #5 materials are then added to the batch. The mixing type and speed are adjusted as necessary as the batch thickens. The Seq. #6 material is then added. The batch is then cooled to 50° C. and the Seq. #7 material is added to the batch. The batch is cooled to 42° C. The Seq. #8 materials are then added, one at a time, mixing well between additions, and reaching uniformity before proceeding. The batch is then cooled to approximately 35° C. The Seq. #9 materials are then added one at a time, mixing well between additions, and reaching uniformity. The batch is then cooled to room temperature, and is preferably compared to a standard.

### EXAMPLE 3

The composition of this example is preferably used as an eye serum and includes the following ingredients as listed in Table 3:

TABLE 3

| Seq. | INCI Name | Trade Name | % w/w |
|---|---|---|---|
| 1 | Water (Aqua) | Deionized Water | 52.71 |
| 2 | Carbomer | Carbopol 940 (BF Goodrich) | 0.30 |
| 3 | Glycerin (and) Water (and) Sodium | Zilgel VV (Presperse) | 20.00 |
| | Polyacrylate | | |
| 4 | Water | Deionized Water | 2.00 |
| 4 | Triethanolamine | TEA 99% | 0.28 |
| 5 | Butylene Glycol | Butylene Glycol | 2.00 |
| 5 | Ethoxydiglycol | Transcutol (Gattefosse) | 0.50 |
| 5 | Salix Alba (Willow) Bark Extract | NAB Willowbark Extract | 0.20 |
| | | #09020 (Arch) | |
| 5 | Hexapeptide-3 | Argireline (Centerchem) | 10.00 |
| 5 | Water (and) Propylene Glycol (and) Citrus | Remoduline (Silab/Rita) | 1.25 |
| | Aurantium Amara (Bitter Orange) Flower | | |
| | Extract | | |
| 5 | Water (and) Sodium Hyaluronate | Hyasol 1% (Centerchem) | 1.00 |
| 5 | Lecithin (and) Magnesium Ascorbyl | Oxysomes (Barnet) | 0.01 |
| | Phosphate (and) Tocopherol | | |
| 6 | Polysorbate 20 | Tween 20 (ISI) | 1.50 |
| 6 | Phenoxyethanol (and) Methylparaben (and) | Phenonip (Nipa) | 1.00 |
| | Propylparaben (and) Ethylparaben (and) | | |
| | Butylparaben | | |
| 6 | Polyethylene | CL-2080 (Kobo) | 1.00 |
| 6 | Isononyl Isononanoate | Dermol 99 (Alzo) | 0.25 |
| 6 | Dimethicone Copolyol | Dow Corning 193 (Dow | 1.00 |
| | | Corning) | |
| 6 | Isostearyl Neopentanoate | Ceraphyl 375 (ISP) | 0.50 |
| 6 | Neopentyl Glycol Dicaprylate/Dicaprate | Liponate NPGC-2 (Lipo) | 0.50 |
| 6 | Cyclomethicone | Dow Corning 345 (Dow | 1.00 |
| | | Corning) | |
| 7 | Water (and) Butylene Glycol (and) Ahnfeltia | APT GC (Centerchem) | 2.00 |
| | Concinna Extract (and) Glycosaminoglycans | | |
| 7 | Imperata Cylindrica (Root) Extract | Moist-24 (Croda) | 1.00 |

With reference to Table 3, the composition of Example 3 is prepared by heating the Seq. #1 water in a stainless steel jacketed kettle equipped with a Lightning® mixer and sweep agitation, to approximately 80-82° C. while agitating to eliminate any possible presence of Gram Positive bacteria. The water is then allowed to cool, and at around 40° C. the Seq. #2 Carbomer is slowly added. The batch is then agitated well and mixed it is lump-free and uniform. Top and bottom samples may be quality checked before proceeding. The Seq. #3 material is then added, switching to sweep as batch thickens to prevent aeration. The batch is allowed to continue cooling. In a side phase stainless steel kettle, the Seq. #4 materials are premixed and added to the batch. The Seq. #5 materials are then added, one at a time, mixing well and preferably reaching uniformity in between additions. In side phase stainless steel kettle, the Seq. #6 materials are premixed and added to the batch. The Seq. #7 materials are added, one at a time, and the batch is mixed until uniform. The batch is allowed to cool to room temperature, and top and bottom samples are taken for comparison to a standard.

### EXAMPLE 4

The composition of this example is preferably used as a facial mask and includes the lowing ingredients as listed in Table 4:

TABLE 4

| Seq. | INCI Name | Trade Name | % w/w |
|---|---|---|---|
| 1 | Water (Aqua) | Water | 78.08 |
| 1 | Methylparaben | Methylparaben | 0.12 |
| 1 | Disodium EDTA | Dissolvine Na2 (Akzo) | 0.10 |
| 1 | Glycerin | Glycerin 96% | 3.00 |
| 2 | Chondrus Crispus | Viscarin SD 389 (FMC) | 0.30 |
| 2 | Chondrus Crispus | Viscarin GP 209 (FMC) | 0.10 |
| 3 | Hydroxyethylcellulose | Natrosol 250HHR | 0.80 |
| | | (Hercules/Aqualon) | |
| 4 | Hypnea Musciformis Extract (and) Gellidiela | Ormagel SH (ISI) | 1.00 |
| | Acerosa Extract (and) Sargassum Filipendula | | |
| | Extract (and) Sorbitol | | |
| 4 | Butylene Glycol (and) Water (Aqua) (and) | Complex EPX-246 BG Anti- | 0.50 |
| | Humulus Lupulus (Hops) Extract (and) | Aging (ISI) | |
| | Rosmarinus Officinalis (and) Equisetum | | |
| | Arvense Extract (and) Pinus Sylvestris Cone | | |
| | Extract (and) Citrus Medica Limonum | | |
| | (Lemon) Fruit Extract | | |
| 4 | Water (and) Hydrolyzed Soy Protein (and) | Oxylastil (Sederma) | 3.00 |
| | Propylene Glycol | | |
| 4 | Hydrolyzed Wheat Protein | Sitolene (Silab/Rita) | 1.00 |
| 4 | Zea Mays (Corn) Extract | Deliner (Coletica Inc.) | 2.00 |
| 4 | Acetyl Hexapeptide-3 | Argireline (Centerchem) | 1.00 |
| 4 | Propylene Glycol (and) Water (and) Butylene | Complex 4-Oxygen | 2.00 |
| | Glycol (and) Vitis Vinifera (Grape) Leaf | (Centerchem) | |
| | Extract (and) Glycine Soya (Soybean) Germ | | |
| | Extract (and) Triticum Vulgare (Wheat) Germ | | |
| | Extract | | |
| 5 | Phenoxythanol (and) Chlorphenesin (and) | Germazide MPB | 1.50 |
| | Glycerin (and) Methylparaben (and) Benzoic | (Collaborative Labs) | |
| | Acid | | |
| 6 | Cucumis Melo (Melon) Extract | Crodarome Liquid Melon | 1.00 |
| | | (Croda) | |
| 6 | Water (and) Butylene Glycol (and) Glycerin | Dragoco Oat Beta Glucan | 2.00 |
| | (and) Avena Sativa (Oat) Kernel Extract | (Dragoco) | |
| 6 | Hydrolyzed Wheat Protein/PVP Crosspolymer | Hydrotriticum PVP (Croda) | 2.50 |

With reference to Table 4, the composition of Example 4 may be prepared by heating the Seq. #1 materials in a jacketed, stainless steel kettle equipped with a variable speed Lightnin® type mixer, to approximately 80° C. while mixing well, to dissolve the methylparaben. The Seq. #2 materials are then added one at a time, into the kettle, under adequate Lightnin® type agitation. One the batch becomes uniform, the materials of Seq. #3 are added as in the previous step. The batch is then cooled to approximately 40° C., switching to sweep setting as the batch thickens, and being careful not to aerate the batch. At approximately 40° C., the Seq. #4 materials are added, one at a time, mixing well in between additions. The batch should preferably be homogeneous prior to each addition. The Seq. # 5 material is then added in the same manner. In a side kettle equipped with Lightnin® type agitation, the Seq. #6 materials are premixed and warmed to 40° C., then added to the batch in the same manner. The batch is mixed until uniform. Top and bottom standards are preferably taken for comparison to a standard.

### EXAMPLE 5

The composition of this example includes the following ingredients as listed in Table 5:

TABLE 5

| Seq. | INCI Name | Trade Name | % w/w |
|---|---|---|---|
| 1 | Water (Aqua) | Water | 62.369 |
| 1 | Glycerin | Glycerin 99% | 4.750 |
| 1 | Disodium EDTA | Dissolvine Na 2 (Akzo) | 0.100 |
| 1 | Methylparaben | Methylparaben | 0.250 |
| 1 | Propylparaben | Propylparaben | 0.100 |
| 2 | Chondrus Crispus (Carrageenan) | Viscarin SD 389 (FMC Corp) | 0.200 |
| 3 | Acrylates/C10-30 Alkyl Acrylate | Carbopol 2020 (Protameen/ | 0.250 |
| | Crosspolymer | BF Goodrich) | |
| 4 | Olive Oil PEG-7 Esters | Olivem 300 (Presperse) | 0.500 |
| 4 | Tridecyl Stearate (and) Neopentyl Glycol | Lipovol MOS-70 (Lipo) | 8.000 |
| | Dicaprylate/Dicaprate (and) Tridecyl | | |
| | Trimellitate | | |
| 4 | Cetearyl Alcohol (and) Ceteareth-20 | Lipowax D (Lipo) | 1.850 |
| 4 | PEG-A Olivate | Olivem 700 (Presperse) | 1.850 |
| 4 | Glyceryl Stearate SE | Lipo 470 SE (Lipo) | 3.750 |
| 4 | Dimethicone | Dow 200 Fluid 350 c/s (Dow | 0.010 |
| | | Corning) | |
| 5 | Polyacrylamide (and) C13-14 Isoparaffin | Sepigel 305 (Seppic) | 1.000 |
| | (and) Laureth-7 | | |
| 6 | Cocamidopropyl Betaine | Cocamidopropyl Betaine | 6.000 |
| | | (Lexaine C) | |
| 7 | Phenoxyethanol | Phenoxyethanol | 1.000 |
| 8 | Glyceryl Polymethacrylate | Lubragel NP (ISP) | 2.000 |
| 9 | Caprylic/Capric Triglyceride (and) Cucumis | Natural Melon Aroma | 2.000 |
| | Melo (Melon) Extract (and) Musa Sapientum | (Ungerer) XX-000981 | |
| | (Banana) Fruit Extract (and) Vitis Vinifera | | |
| | (Grape) Fruit Extract (and) Iris Florentina | | |
| | Root Extract (and) Hibiscus Abelmoschuus | | |
| 9 | Cucumis Melo (Melon) Extract | Liquid Melon Crodarome | 1.000 |
| | | (Croda) | |
| 9 | Water (and) Butylene Glycol (and) Ahnfeltia | APT GC (Centerchem) | 0.010 |
| | Concinna Extract (and) Glycosaminoglycans | | |
| 9 | Isododecane | Permethyl 99A (Presperse) | 3.000 |
| 9 | Vitis Vinifera (Grape) Seed Extract (and) | Leucocyandin Phytosome | 0.001 |
| | Phospholipids | (Indena - Lipo) | |
| 9 | Acetyl Hexapeptide-3 | Argireline (Centerchem) | 0.010 |

With reference to Table 5, the composition of Example 5 is prepared by heating the combined Seq. #1 materials in a stainless steel, jacketed main kettle, equipped with sweep agitation and a Lightnin® mixer to approximately 78-80° C. under Lightnin® mixing. The Seq. #2 material is then sprinkled slowly and evenly across the agitating batch. Care should be taken not to aerate the batch. The batch is then checked to make sure that it is lump free and cooled to approximately 76-78° C. The Seq. #3 material is then added in a similar manner. In a stainless steel side kettle with good agitation, the Seq. #4 materials are combined and heated to approximately 80-82° C. At proper temperatures, the Seq. #4 batch is slowly added to the main kettle, while maintaining good agitation and temperature, until the emulsion is well formed and stabilized. The batch is then cooled to approximately 70° C. At approximately 70° C., the Seq. #5 material is added. This material should be well mixed prior to adding, since it may separate in storage. At this point it may be necessary to increase the mixing speed until batch is smooth and uniform. The prior mixing speed may then be resumed, or the sweep setting may be used. The Seq. #6 then the Seq. #7 materials are then added. The Seq. #8 material is then added, being sure that the material is evenly dispersed before continuing. The batch is then cooled to approximately 42° C. and the aromatic Seq. #9 materials are added. The batch is then cooled to o approximately 35° C., and the remaining Seq. #9 materials are added. The batch should be mixed until evenly distributed. The Seq. #9 materials may be added one at a time, or a premix may be created for addition. The batch is allowed to cool to 25° C. and top and bottom samples may be taken and compared to a standard.

### EXAMPLE 6

The composition of this example may be used as an exfoliant, and includes the following ingredients as listed in Table 6. (The table provides alternative weight percentages, % w/w₁ and % w/w₂, which may be used in accordance with first and second alternative embodiments).

TABLE 6

| Seq. | INCI Name | Trade Name | % w/w1 | % w/w2 |
|---|---|---|---|---|
| 1 | Water (Aqua) | Water | 47.545 | 56.479 |
| 1 | Glycerin | Glycerin 99% | 4.750 | 4.750 |
| 1 | Disodium EDTA | Dissolvine Na 2 (Akzo) | 0.100 | 0.100 |
| 1 | Methylparaben | Methylparaben | 0.250 | 0.250 |
| 1 | Propylparaben | Propylparaben | 0.100 | 0.100 |
| 2 | Chondrus Crispus (Carrageenan) | Viscarin SD 389 (FMC | 0.250 | 0.250 |
| | | Corp) | | |
| 3 | Xanthan Gum | Keltrol CG (Kelco) | 0.150 | 0.150 |
| 4 | Olive Oil PEG-7 Esters | Olivem 300 (Presperse) | 0.900 | 0.500 |
| 4 | Tridecyl Stearate (and) Neopentyl Glycol | Lipovol MOS-70 (Lipo) | 14.000 | 10.000 |
| | Dicaprylate/Dicaprate (and) Tridecyl | | | |
| | Trimellitate | | | |
| 4 | Cetearyl Alcohol (and) Ceteareth-20 | Lipowax D (Lipo) | 3.000 | 3.000 |
| 4 | PEG-4 Olivate | Olivem 700 (Presperse) | 1.850 | 1.850 |
| 4 | Glyceryl Stearate SE | Lipo 470 SE (Lipo) | 3.750 | 3.750 |
| 4 | Dimethicone | Dow 200 Fluid 350 c/s | 0.450 | 0.050 |
| | | (Dow Corning) | | |
| 5 | Polyacrylamide (and) C13-14 Isoparaffin | Sepigel 305 (Seppic) | 0.750 | 0.750 |
| | (and) Laureth-7 | | | |
| 6 | Disodium Laureth Sulfosuccinate (and) | Chemoryl SFB-2 | 7.000 | 6.000 |
| | Cocamidopropyl Hydroxysultaine (and) | (Chemron) | | |
| | Myristamine Oxide (and) Glycerin (and) | | | |
| | Sodium Cocoyl Glutamate (and) PEG-150 | | | |
| | Distearate | | | |
| 7 | Phenoxyethanol | Phenoxyethanol | 1.000 | 1.000 |
| 8 | Glyceryl Polymethacrylate | Lubragel NP (ISP) | 2.200 | 2.000 |
| 8 | Caprylic/Capric Triglyceride (and) | Natural Melon Aroma | 1.000 | 1.000 |
| | Cucumis Melo (Melon) Extract (and) | (Ungerer) XX-000981 | | |
| | Musa Sapientum (Banana) Fruit Extract | | | |
| | (and) Vitis Vinifera (Grape) Fruit Extract | | | |
| | (and) Iris Florentina Root Extract (and) | | | |
| | Hibiscus Abelmoschuus | | | |
| 8 | Cucumis Melo (Melon) Extract | Liquid Melon Crodarome | 1.000 | 1.000 |
| | | (Croda) | | |
| 9 | Water (and) Butylene Glycol (and) | APT GC (Centerchem) | 0.100 | 0.010 |
| | Ahnfeltia Concinna Extract (and) | | | |
| | Glycosaminoglycans | | | |
| 10 | Isododecane | Permethyl 99A (Presperse) | 4.750 | 3.000 |
| 10 | Vitis Vinifera (Grape) Seed Extract (and) | Leucocyandin Phytosome | 0.005 | 0.001 |
| | Phospholipids | (Indena - Lipo) | | |
| 11 | Acetyl Hexapeptide-3 | Argireline (Centerchem) | 0.100 | 0.010 |
| 11 | Salix Alba (Willow) Bark Extract | NAB Willow bark Extract | 1.000 | 1.000 |
| | | #09020 (Arch) | | |
| 11 | Hydrogenated Jojoba Wax | Ice White Jojoba Spheres | 2.000 | 2.000 |
| | | 40/60 (Desert Whale | | |
| 11 | Lithothamnium Clacarum Powder | Exfosea Lithothamnium | 2.000 | 1.000 |
| | | Blue/Gray (Barnet) | | |

With reference to Table 6, the composition of Example 6 is prepared by heating the combined Seq. #1 materials in a stainless steel, jacketed main kettle, equipped with sweep agitation and a Lightnin® mixer to approximately 78-80° C. under Lightnin® mixing. The Seq. #2 material is then sprinkled slowly and evenly across the agitating batch. Care should be taken not to aerate the batch. The batch is then checked to make sure that it is lump free and cooled to approximately 76-78° C. The Seq. #3 material is then added in a similar manner. In a stainless steel side kettle with good agitation, the Seq. #4 materials are combined and heated to approximately 80-82° C. At proper temperatures, the Seq. #4 batch is slowly added to the main kettle, while maintaining good agitation and temperature, until the emulsion is well formed and stabilized. The batch is then cooled to approximately 70° C., switching to sweep as the batch thickens. At approximately 70° C., the Seq. #5 material is added. This material should be well mixed prior to adding, since it may separate in storage. At this point it may be necessary to increase the mixing speed until batch is smooth and uniform. The prior mixing speed may then be resumed, or the sweep setting may be used. The batch is then cooled to approximately 45° C., and the Seq. #6 then the Seq. #7 materials are added. The Seq. #8 material is then added, being sure that the material is evenly dispersed before continuing. The batch is then cooled to approximately 42° C. and the aromatic Seq. #9 materials are added, one at a time, mixing until the batch is uniform in between additions. The batch is then cooled to approximately 35° C., and the remaining Seq. #9 materials are added. The batch should be mixed until evenly distributed. The Seq. #10 materials are then added, one at a time, mixing well with each addition. The batch is mixed until uniform. The Seq. #11 materials are then added, and the batch is mixed until the particles are evenly distributed. The batch is allowed to cool to 25° C. and top and bottom samples may be taken and compared to a standard.

### EXAMPLE 7

The composition of this example is preferably used as a skin refining primer and includes the following ingredients as listed in Table 7:

TABLE 7

| Seq. | INCI Name | Trade Name | % w/w |
|---|---|---|---|
| 1 | Water (Aqua) | Deionized Water | 83.195 |
| 1 | Glycerin | Glycerin | 3.000 |
| 1 | Disodium EDTA | Dissolvine Na2 (Akzo) | 0.10 |
| 1 | Aloe Barbadensis Leaf Juice | Aloe Barbadensis Leaf Juice | 1.00 |
| | | (Lily of the Desert) | |
| 1 | Sodium PCA | Ajidew N-50 (Ajinomoto) | 0.500 |
| 1 | Methylparaben | Methylparaben | 0.250 |
| 1 | Benzophenone-4 | Benzophenone-4 | 0.050 |
| 1 | Butylene Glycol | Butylene Glycol | 3.000 |
| 2 | Hydroxyethylcellulose | Natrosol HHR | 0.750 |
| | | (Hercules/Aqualon) | |
| 3 | Salix Alba (Willow)Bark Extract | NAB Willowbark Extract | 3.500 |
| | | #09020 (Arch) | |
| 4 | Lavendula Angustifolia (Lavender) oil | Lavender Oil (Ungerer) | 0.020 |
| 4 | Propylparaben | Propylparaben | 0.020 |
| 4 | PPG-5-Ceteth-20 | Procetyl AWS (Croda) | 1.500 |
| 5 | Water (and) butylene glycol (and) Camellia | Actiphyte of Green Tea BG50 | 0.100 |
| | Oleifera Leaf Extract | (Active Organics) | |
| 6 | Phenoxyethanol | Phenoxyethanol | 1.000 |
| 7 | Water (and) Butylene Glycol (and) Ahnfeltia | APT GC (Centerchem) | 2.000 |
| | Concinna Extract (and) Glycosaminoglycans | | |
| 8 | Water (and) Red No. 40 (CI 16035) | Red No. 40(1% Aq.Sol) | 0.015 |

With reference to Table 7, the composition of Example 7 is prepared by heating the Seq. #1 materials in a stainless steel jacketed kettle equipped with a Lightning® mixer and sweep agitation, to approximately 80° C. while mixing to solublize the methylparaben and eliminate any gram Positive bacteria that may be present. The water is then cooled to 45° C. During the cooling process, the Seq. #2 material may be slowly incorporated to the kettle, while under adequate agitation. Top and bottom samples from the batch are checked for lumps before proceeding. The Seq. #3 material is then added. In a stainless steel side kettle equipped with Lightnin® type agitation, the Seq. #4 materials are premixed. It may be necessary to warm the mixture to approximately 40-42° C. in order to obtain clarity. Very slowly, and under high energy, the Seq. #4 mixture is added to the main batch. The Seq. #4 mixture is preferably added in small intervals, wherein the batch is allowed to clear before additions, in order to obtain a clear batch. The Seq. #5, 6, and 7 materials are added in the same manner, one at a time, being sure that the batch is clear and uniform in between additions. The Seq. #8 material is then added to the batch. Top and bottom samples may be taken for comparison to a standard.

### EXAMPLE 8

The composition of this example includes the following ingredients as listed in Table 8:

TABLE 8

| Sequence | INCI Name | Trade Name | % W/W |
|---|---|---|---|
| 1 | Water (Aqua) | Water | 67.971 |
| 1 | Disodium EDTA | Dissolvine Na2 | 0.100 |
| | | (Akzo) | |
| 2 | Carrageenan (Chondrus Crispus) | Viscarin SD 389 | 1.400 |
| 3 | Glycerin (and) Glyceryl | Hispagel 200 | 14.000 |
| | Polyacrylate | | |
| 4 | Glycerin (and) Butylene Glycol | APT/GC | 2.000 |
| | (and) Water (Aqua) Ahnfeltia | (Centerchem) | |
| | Concinna extract | | |
| 5 | Acetyl Hexapeptide-3 | Argireline | 5.000 |
| 6 | Ceramide 3 (and) Ceramide 3 (B) (and) | SK-Influx | 5.000 |
| | Ceramide 6-II (and) | | |
| | Ceramide I (and) Cholesterol | | |
| | (and) Phytosphingosine (and) | | |
| | Carbomer (and) Xanthan Gum | | |
| | (and) Methylparaben (and) Propylparaben | | |
| 6 | Benzophenone-4 | Uvinul MS-40 BASF | 0.020 |
| 7 | Phenoxyethanol, Methylparaben, | Phenonip | 1.000 |
| | Propylparaben, Ethylparaben, | | |
| | Butylparaben | | |
| 7 | Caprylic/Capric Triglyceride | Natural Melon | 0.500 |
| | (and) Cucumis Melo (Melon) | Concentrate #51226 | |
| | extract (and) Musa Sapientum | (Belle Aire) | |
| | (Banana) Fruit Extract (and) Vitis | | |
| | Vinifera (Grape) Fruit extract | | |
| | (and) Iris Florentina | | |
| 8 | Water, Blue No. 1 (CI 42090) | Blue #1,; 1.00% Aq. | 0.006 |
| | | Sol. | |

A cleanser composition in accordance with the present invention may also include the following list of ingredients: APT, Willow Bark Extract, and Hexapeptide-3, water, cocamidopropyl betaine, Laureth-2, Cocamine oxide, Polysorbate 20, Cucumis Melo Extract, Caprylic/Capric Triglyceride, Musa Sapientum Fruit Extract, grape fruit extract, Iris Florentina Root extract, Hibiscus Abelmoschuus, Panthenol, grape seed extract, phospholipids, Allantoin, Lecithin, Magnesium ascorbyl phosphate, Tocopherol, Cabomer, Triethanolamine, Sodium Sulfite, Phenoxyethanol, Methylparaben, Propylparaben, and Disodium EDTA.

Though the term “batch” is used in the above examples, it is to be understood that the compositions may be formed in batch or continuous processes.

In closing, it is to be understood that the exemplary embodiments described herein are illustrative of the principles of the present invention. Other modifications that may be employed are within the scope of the invention. Thus, by way of example, but not of limitation, alternative configurations may be utilized in accordance with the teachings herein. Accordingly, the description is illustrative and not meant to be a limitation thereof.

1. A skin care composition, comprising:
a safe and effective amount of at least one anti-wrinkling agent and
a safe and effective amount of a natural exfoliating complex.

2. The skin care composition of claim 1 wherein the anti-wrinkling agent is a hexapeptide.

3. The skin care composition of claim 2 wherein the hexapeptide is an acetyl hexapeptide.

4. The skin care composition of claim 1 wherein the natural exfoliating complex comprises Ahnfeltia cocinna.

5. The skin care composition of claim 4 wherein the natural exfoliating complex further comprises butylene glycol and glycosaminoglycans.

6. The skin care composition of claim 1 further comprising one or more components selected from the group consisting of conditioning agents, skin protectants, other antioxidants, UV absorbing agents, sunscreen actives, cleansing agents, viscosity modifying agents, film formers, emollients, surfactants, solubilizing agents, preservatives, fragrance, chelating agents, foaming or antifoaming agents, opacifying agents, stabilizing agents, pH adjustors, absorbents, anti-caking agents, slip modifiers, various solvents, solubilizing agents, denaturants, abrasives, bulking agents, emulsion stabilizing agents, suspending agents, colorants, binders, conditioning agent-emollients, surfactant emulsifying agents, biological products, anti-acne actives, anti-wrinkle and anti-skin atrophy actives, skin barrier repair aids, cosmetic soothing aids, topical anesthetics, artificial tanning agents and accelerators, skin lightening actives, antimicrobial and antifingal actives, sebum stimulators, sebum inhibitors, humectants, and combinations thereof.

7. A skin care composition, comprising:
a safe and effective amount of at least one hexapeptide; and
a safe and effective amount of a natural exfoliating complex comprising Ahnfeltia cocinna, wherein the skin care composition provides for natural skin exfoliation, reduces fine lines and wrinkles, and improves skin elasticity and firmness.

8. The skin care composition of claim 7 wherein the hexapeptide is an acetyl hexapeptide.

9. The skin care composition of claim 7 wherein the complex further comprises butylene glycol and glycosaminoglycans.

10. The skin care composition of claim 7 further comprising one or more components selected from the group consisting of conditioning agents, skin protectants, other antioxidants, UV absorbing agents, sunscreen actives, cleansing agents, viscosity modifying agents, film formers, emollients, surfactants, solubilizing agents, preservatives, fragrance, chelating agents, foaming or antifoaming agents, opacifying agents, stabilizing agents, pH adjustors, absorbents, anti-caking agents, slip modifiers, various solvents, solubilizing agents, denaturants, abrasives, bulking agents, emulsion stabilizing agents, suspending agents, colorants, binders, conditioning agent-emollients, surfactant emulsifying agents, biological products, anti-acne actives, anti-wrinkle and anti-skin atrophy actives, skin barrier repair aids, cosmetic soothing aids, topical anesthetics, artificial tanning agents and accelerators, skin lightening actives, antimicrobial and antifungal actives, sebum stimulators, sebum inhibitors, humectants, and combinations thereof.

11. The skin care composition of claim 7 wherein the hexapeptide has a concentration ranging from approximately 0.01% w/w to approximately 15% w/w.

12. The skin care composition of claim 7 wherein the Ahnfeltia cocinna has a concentration ranging from approximately 0.01% w/w to approximately 5% w/w.

13. A skin care composition, comprising:
safe and effective amounts of an acetyl hexapeptide-3, Ahnfeltia cocinna, butylene glycol, and glycosaminoglycans, wherein the skin care composition provides for natural skin exfoliation, reduces fine lines and wrinkles, and improves skin elasticity and firmness.

14. The skin care composition of claim 13 further comprising one or more components selected from the group consisting of conditioning agents, skin protectants, other antioxidants, UV absorbing agents, sunscreen actives, cleansing agents, viscosity modifying agents, film formers, emollients, surfactants, solubilizing agents, preservatives, fragrance, chelating agents, foaming or antifoaming agents, opacifying agents, stabilizing agents, pH adjustors, absorbents, anti-caking agents, slip modifiers, various solvents, solubilizing agents, denaturants, abrasives, bulking agents, emulsion stabilizing agents, suspending agents, colorants, binders, conditioning agent-emollients, surfactant emulsifying agents, biological products, anti-acne actives, anti-wrinkle and anti-skin atrophy actives, skin barrier repair aids, cosmetic soothing aids, topical anesthetics, artificial tanning agents and accelerators, skin lightening actives, antimicrobial and antifungal actives, sebum stimulators, sebum inhibitors, humectants, and combinations thereof.

15. The skin care composition of claim 13 wherein the hexapeptide has a concentration ranging from approximately 0.01% w/w to approximately 15% w/w.

16. The skin care composition of claim 14 wherein the Ahnfeltia cocinna has a concentration ranging from approximately 0.01% w/w to approximately 5% w/w.

17. A method for providing natural skin exfoliation and wrinkle reduction, comprising:
providing a skin care composition comprising a safe and effective amount of at least one hexapeptide, and a safe and effective amount of a natural exfoliating complex comprising Ahnfeltia cocinna; and
applying the skin care composition to a portion of an individual's skin.

18. A skin care composition comprising:
a safe and effective amount of a hexapeptide; and
at least one ingredient selected from a group comprising Willow Bark Extract, APT, Moist 24, Skin Flux, Polyolprepolymer-15, Dipalmitoyl Hydroxyproline, zea mays extract, and Oat Beta Glucan.

19. A skin care composition comprising at least three ingredients selected from the following group: a hexapeptide complex, Willow Bark Extract, APT, Moist 24, Skin Flux, Polyolprepolymer-15, Dipalmitoyl Hydroxyproline, zea mays extract, and Oat Beta Glucan.
